(19) United States
(12) Patent Application Publication
(10) Pub. No.: US 2024/0000471 A1
(43) Pub. Date: Jan. 4, 2024

(54) FORCEPS WITH DEFLECTABLE GRASPING PLATFORMS

(71) Applicant: XRV-IP, LLC, Chesterfield, MO (US)

(72) Inventor: James C. Easley, Cottleville, MO (US)

(21) Appl. No.: 18/344,465

(22) Filed: Jun. 29, 2023

Related U.S. Application Data
(60) Provisional application No. 63/367,469, filed on Jun. 30, 2022.

(51) Int. Cl. A61B 17/29

(52) U.S. Cl. CPC A61B 17/29; A61B 2017/2932; A61B 2017/2902

(57) ABSTRACT

A forceps includes a forceps tip including a first arm and a second arm. The first arm includes a first grasping platform including a first arcuate inner engagement surface. The second arm includes a second grasping platform including a second arcuate inner engagement surface. Each of the first and second engagement surfaces is arcuate about a transverse axis extending between the first and second engagement surfaces and perpendicular to a longitudinal axis of a forceps handle. The first and second grasping platforms are configured to initially contact one another only at their respective distal ends along the first and second engagement surfaces when actuated from an open position to a closed position, and are configured to deflect such that a contact area between the first and second engagement surfaces increases as the first and second grasping platforms move towards the closed position.

### CROSS-REFERENCE TO RELATED APPLICATIONS

This application claims priority to U.S. Provisional Patent Application No. 63/367,469, filed Jun. 30, 2022, the disclosure of which is hereby incorporated by reference in its entirety.

### BACKGROUND

The field of the disclosure relates generally to a forceps for manipulating tissue, and, more particularly, to a forceps with deflectable grasping platforms.

Forceps have a wide range of applications for manipulating tissue across many medical specialties. In ophthalmology, and particularly in procedures in the posterior segment of the eye, styles of forceps have been developed to address the need of manipulating very fine, thin membranes. In these types of procedures, it is desirable to be able to ensure a firm grip on the membrane with minimal access to the membrane. The grip needs to be obtained without applying too much compression to the membrane, which can cause the membrane to tear or shred.

Many conventional forceps rely on two grasping platforms that are flat and stiff. The grasping platforms are approximated or closed against each other to grasp whatever is placed between them. The closing means used to move the grasping platforms into approximation influences how much pressure is placed on the grasping platforms and how the pressure is distributed across the grasping platforms. In general, a stiffer closing means enables better control of the position of the grasping platforms relative to each other at the expense of total pressure control applied between the grasping platforms. As varying thicknesses of tissue are placed between the grasping platforms, the pressure applied to the tissue by the grasping platforms can vary significantly. Additionally, minor errors in calibration of the closing means can cause significant pressure variations. A closing means with greater flexibility allows the total applied pressure to vary less with tissue thickness. However, pressure distribution across the grasping platforms is less consistent with a closing means having greater flexibility. The flexibility tends to allow the grasping platforms to deviate from the parallel relationship that yields even pressure distribution across the planar platform surface.

Accordingly, there is a need for a forceps that enables better pressure distribution between the grasping platforms over the tissue, and that can facilitate reducing or preventing structural damage to the tissue from the grasping platforms.

### BRIEF DESCRIPTION

In one aspect, a forceps includes a handle extending along a longitudinal axis and including an actuator, an actuation tube operatively coupled to the actuator, and a forceps tip. The forceps tip includes a first arm and a second arm. The first arm includes a first grasping platform extending from a proximal end to a distal end. The first grasping platform includes a first inner engagement surface that extends arcuately from the proximal end to the distal end. The second arm includes a second grasping platform extending from a proximal end to a distal end. The second grasping platform includes a second inner engagement surface that extends arcuately from the proximal end to the distal end. Each of the first and second engagement surfaces is arcuate about a transverse axis extending between the first and second engagement surfaces and perpendicular to the longitudinal axis of the handle. Actuation of the actuator causes the actuation tube to move distally from the handle and into engagement with the first and second arms to cause the first and second grasping platforms to move from an initial, open position towards a closed position. The first and second grasping platforms are configured to initially contact one another only at their respective distal ends along the first and second engagement surfaces when actuated from the open position to the closed position. Each of the first and second grasping platforms is configured to deflect such that a contact area between the first and second engagement surfaces increases from their respective distal ends to their respective proximal ends as the first and second grasping platforms move towards the closed position.

In another aspect, a forceps includes a handle extending along a longitudinal axis and including an actuator, and a forceps tip operatively coupled to the actuator. The forceps tip includes a first arm and a second arm. The first arm includes a first grasping platform extending from a proximal end to a distal end. The first grasping platform includes a first inner engagement surface that extends arcuately from the proximal end to the distal end. The second arm includes a second grasping platform extending from a proximal end to a distal end. The second grasping platform includes a second inner engagement surface that extends arcuately from the proximal end to the distal end. Actuation of the actuator causes the first and second grasping platforms to move from an initial, open position towards a closed position. Each of the first and second grasping platforms is configured to deflect such that a contact area between the first and second engagement surfaces continuously increases between their respective distal ends and their respective proximal ends as the first and second grasping platforms move towards the closed position.

In yet another aspect, a method of operating forceps includes providing a forceps. The forceps includes a handle extending along a longitudinal axis and including an actuator, an actuation tube operatively coupled to the actuator, and a forceps tip. The forceps tip includes a first arm and a second arm. The first arm includes a first grasping platform extending from a proximal end to a distal end. The first grasping platform including a first inner engagement surface that extends arcuately from the proximal end to the distal end. The second arm includes a second grasping platform extending from a proximal end to a distal end. The second grasping platform includes a second inner engagement surface that extends arcuately from the proximal end to the distal end. Each of the first and second engagement surfaces is arcuate about a transverse axis extending between the first and second engagement surfaces and perpendicular to the longitudinal axis of the handle. The method further includes actuating the actuator such that the actuation tube moves distally from the handle and into engagement with the first and second arms to cause the first and second grasping platforms to move from an initial, open position towards a closed position. Actuating the actuator causes the first and second grasping platforms to initially contact one another only at their respective distal ends along the first and second engagement surfaces when actuated from the open position to the closed position. Actuating the actuator causes each of the first and second grasping platforms to deflect such that a contact area between the first and second engagement surfaces increases from their respective distal ends to their respective proximal ends as the first and second grasping platforms move towards the closed position.

Various refinements exist of the features noted in relation to the above-mentioned aspects of the present disclosure. Further features may also be incorporated in the above-mentioned aspects of the present disclosure as well. These refinements and additional features may exist individually or in any combination. For instance, various features discussed below in relation to any of the illustrated embodiments of the present disclosure may be incorporated into any of the above-described aspects of the present disclosure, alone or in any combination.

### BRIEF DESCRIPTION OF THE DRAWINGS

FIG. 1 is a perspective view of an example forceps including a forceps tip with non-planar or curved grasping platforms.

FIG. 2 is a partially exploded perspective view of the forceps tip and an actuation tube of the forceps of FIG. 1.

FIG. 3 is a perspective view of a portion of the forceps tip and actuation tube shown in FIG. 2.

FIG. 4 is a top view of the forceps tip and actuation tube shown in FIG. 3.

FIG. 5 is a side view of the forceps tip and actuation tube shown in FIG. 3.

FIG. 6A is an enlarged top view of the forceps tip and actuation tube shown in FIG. 3 illustrating the forceps tip in an initial, open position.

FIG. 6B is another enlarged top view of the forceps tip and actuation tube shown in FIG. 3 illustrating the response of the forceps tip to actuation via the actuation tube.

FIG. 6C is another enlarged top view of the forceps tip and actuation tube shown in FIG. 3 illustrating the forceps tip in a fully actuated or closed position.

FIG. 7 is an enlarged top view of the forceps tip shown in FIG. 6A illustrating the grasping platforms in the initial, open position.

FIG. 8 is an enlarged top view of the forceps tip shown in FIG. 6B illustrating the response of the grasping platforms to actuation.

FIG. 9 is another enlarged top view of the forceps tip shown in FIG. 8 illustrating the response of the grasping platforms to further actuation.

FIG. 10 is an enlarged top view of the forceps tip shown in FIG. 6C illustrating the response of the grasping platforms in the fully actuated or closed position.

FIG. 11 is a perspective view of a first comparative forceps tip and an actuation tube.

FIG. 12 is a top view of the forceps tip and actuation tube shown in FIG. 11.

FIG. 13 is a side view of the forceps tip and actuation tube shown in FIG. 11.

FIG. 14A is an enlarged top view of the forceps tip and actuation tube shown in FIG. 11, with the forceps tip shown in an open position.

FIG. 14B is another enlarged top view of the forceps tip and actuation tube shown in FIG. 14A illustrating the response of the forceps tip to actuation, with the forceps tip shown in a partially closed position.

FIG. 14C is another enlarged top view of the forceps tip and actuation tube shown in FIG. 14B illustrating the response of the forceps tip to further actuation, with the forceps tip shown in a fully closed position.

FIG. 15 is an enlarged top view of the forceps tip shown in FIG. 14A.

FIG. 16 is an enlarged top view of the forceps tip shown in FIG. 14B.

FIG. 17 is another enlarged top view of the forceps tip shown in FIG. 16 illustrating the response of the grasping platforms to further actuation.

FIG. 18 is an enlarged top view of the forceps tip shown in FIG. 14C.

FIG. 19 is a perspective view of a second comparative forceps tip and actuation tube.

FIG. 20 is a top view of the forceps tip and actuation tube shown in FIG. 19.

FIG. 21 is a side view of the forceps tip and actuation tube shown in FIG. 19.

FIG. 22A is an enlarged top view of the forceps tip and actuation tube shown in FIG. 19 illustrating the response of the forceps tip to actuation, with the forceps tip shown in an open position.

FIG. 22B is another enlarged top view of the forceps tip and actuation tube shown in FIG. 22A illustrating the response of the forceps tip to actuation.

FIG. 22C is another enlarged top view of the forceps tip and actuation tube shown in FIG. 22B illustrating the response of the forceps tip to further actuation, with the forceps tip shown in a fully closed position.

FIG. 23 is an enlarged top view of the forceps tip shown in FIG. 22A.

FIG. 24 is an enlarged top view of the forceps tip shown in FIG. 22B.

FIG. 25 is another enlarged top view of the forceps tip shown in FIG. 22C.

### DETAILED DESCRIPTION

The present disclosure is directed to a forceps that is particularly suitable for use in manipulation of tissue. Embodiments of the forceps described herein include a forceps tip that includes two grasping platforms. Embodiments of the grasping platforms described herein include grasping platforms that are curved (i.e., non-planar) and flexible or deflectable. The grasping platforms are oriented such that distal ends of the grasping platforms converge towards each other as the grasping platforms are moved towards each other. In other words, as the grasping platforms converge towards each other, the grasping platforms are not parallel to each other, but rather approach each other more closely on the distal ends than they do on proximal ends. An inner engagement surface of each grasping platform has a curved, cylindrical shape. Additionally, an outer surface of each grasping platform tapers away from the inner surface to give the platform an increase in thickness between the distal end and the proximal end of each grasping platform. In some embodiments, the taper rate of the outer surface is sufficient to allow each grasping platform to flex or deflect proportional to the curvature of its inner surface as pressure is applied to the grasping platforms.

Actuation (i.e., closing) of the forceps tip causes the distal ends of the grasping platforms to make contact first, which forms a wedge-shaped gap on the proximal side of the grasping platforms. As actuation continues, each grasping platform articulates (e.g., flexes) to bring the more distal portions of the grasping platforms together to form a flat contact area between the inner engagement surfaces of the grasping platforms. This action causes the rising force formed between the grasping platforms (from the actuation movement) to spread across the contact area, and thereby create a pressure distribution. As actuation continues further, the articulation of the grasping platforms also continues, and the contact area increases. This, again, spreads the rising force over the larger contact area between the grasping platforms. When the grasping platforms are fully articulated, they are substantially flat, with the entire inner engagement surface of each grasping platform in contact with each other. Upon full articulation, force between the grasping platforms is at its highest and the contact area between the grasping platforms is also at its highest. Such a configuration enables a better distribution of the pressure formed between the grasping platforms over the tissue, and can help to avoid structure damage to the tissue from the grasping platforms.

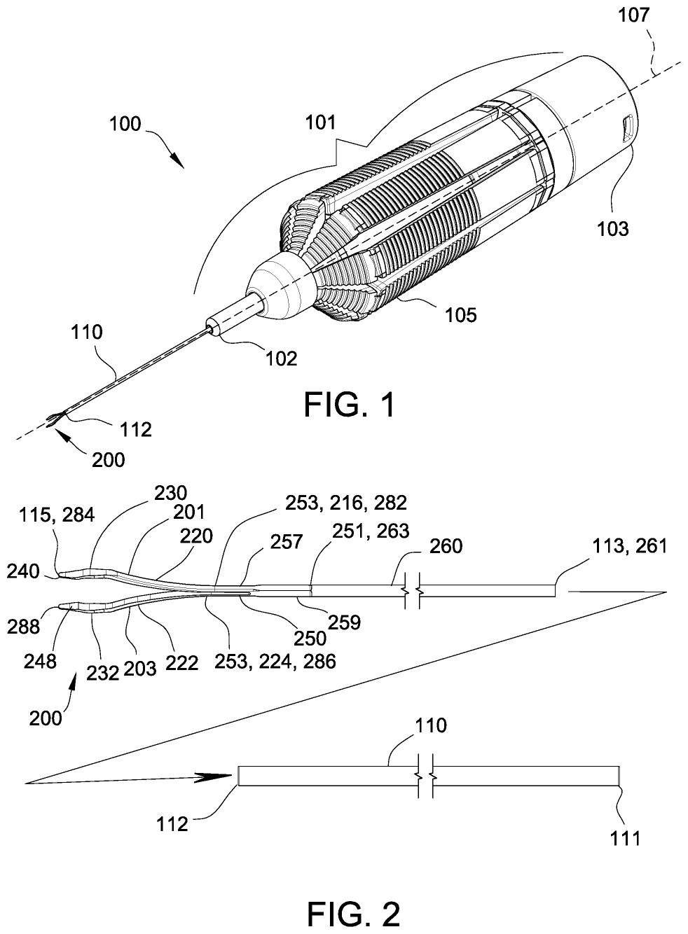

FIG. 1 is a perspective view of an example forceps 100 that includes a forceps tip 200 with non-planar or curved, deflectable grasping platforms, described in further detail herein. As illustrated in FIG. 1, the forceps 100 includes a handle 101, an actuation tube 110, and the forceps tip 200. The handle 101 extends along a longitudinal axis 107 from a proximal end 103 to a distal end 102. The handle 101 also includes an actuator 105, also referred to herein as a control. In other embodiments, the handle 101 may be substituted with other known handles that enable the forceps 100 to function as described herein.

With additional reference to FIG. 2, the forceps tip 200 extends from a proximal end 113 to a distal end 115 and is operatively coupled to the actuator 105 (e.g., via the actuation tube 110). The proximal end 113 of the forceps tip 200 is fixed relative to the proximal end 103 of the handle 101 (e.g., via anchor wire 260). In the example embodiment, a proximal portion (not shown) of the forceps tip 200 is disposed within the actuation tube 110. The actuation tube 110 is operatively coupled to the actuator 105 such that the actuation tube 110 reciprocates axially (i.e., in a proximal direction or in a distal direction—along the axis 107) over a controlled distance when actuated by the actuator 105 (e.g., via a push button or switch). In the example embodiment, the actuator 105 provides for 360 degrees of actuation. That is, the actuator 105 can be actuated in any rotational position of the handle 101 relative to axis 107 (i.e., rotation about the axial direction). As described further herein, actuation of the actuator 105 causes the actuation tube 110 to move distally from the handle 101 along longitudinal axis 107 and to engage the forceps tip 200 to thereby move the forceps tip 200 from an open position to a closed position.

In some embodiments, the forceps 100 is particularly configured (i.e., sized, shaped, constructed of materials with suitable stiffness, biocompatibility, etc.) for performing ophthalmic procedures. In other embodiments, the forceps 100 may configured for use in any other type of procedure.

As shown in FIG. 2, the actuation tube 110 extends from a proximal end 111 to a distal end 112. In the embodiment shown in FIG. 2, the actuation tube 110 is a hollow tube. Suitable materials from which the actuation tube may be constructed include, for example and without limitation, stainless steel. However, other medical grade materials could also be used to construct the actuation tube 110, and the actuation tube 110 may also be of any suitable configuration that enables the forceps 100 to function as described herein.

The forceps tip 200 includes an anchor wire 260, a ramp terminus 250, a first arm 201, and a second arm 203. The anchor wire 260 extends from a proximal end 261 to a distal end 263 and through the actuation tube 110. The anchor wire 260 is fixed to the handle 101 such that the forceps tip 200 is fixed relative to the handle 101. In the illustrated embodiment, each of the first and second arms 201, 203 is coupled to the anchor wire 260 (e.g., via ramp terminus 250). The ramp terminus 250 extends from a proximal end 251 connected to the distal end 263 of the anchor wire 260 to a split distal end 253. In the embodiment shown in FIG. 2, the ramp terminus 250 includes a distal split portion 257 and a proximal non-split portion 259. In the split portion 257, the ramp terminus 250 is split into two extension members from which the first arm 201 and the second arm 203 extend. In other embodiments, the ramp terminus 250 may be entirely split, entirely non-split, or may be of any other suitable configuration that enables the forceps 100 to function as described herein.

The first arm 201 extends from a proximal end 282 to a distal end 284 and includes a first ramp portion 220, a first intermediate portion 230, and a first grasping platform 240. The second arm 203 extends from a proximal end 286 to a distal end 288 and includes a second ramp portion 222, a second intermediate portion 232, and a second grasping platform 248. In some embodiments, the ramp terminus 250 may be formed from part of the first and/or second arms 201, 203. As shown in FIG. 2, the first and second grasping platforms 240, 248 are disposed at the distal end 115 of the forceps tip 200.

The forceps tip 200 may be constructed from a variety of materials. In some embodiments, for example, the forceps tip 200 is formed from a medical grade material such as stainless steel, titanium, or nickel-titanium alloy. In other embodiments, the forceps tip 200 may be formed from a medical grade polymer, a composite, or a ceramic. By way of example, the forceps tip 200 can be manufactured or formed using wire electric discharge machining (EDM), laser cutting, stamping, forming, and molding. In yet other embodiments, additive manufacturing methods, including laser sintering, may be used to manufacture the forceps tip 200. In FIG. 2, the forceps tip 200 is shown as constructed from a single piece of material. That is, the anchor wire 260 and the first and second arms 201, 203 (including the ramp terminus 250) are integrally formed from a single piece of material. However, in other embodiments, the forceps tip 200 may be constructed from multiple pieces of material (e.g., to potentially reduce cost). For example, the anchor wire 260 can be formed from a separate piece of material that is attached near the ramp terminus 250. As an additional example, the forceps tip 200 may also be split along an axis (e.g., along longitudinal axis 107 of the forceps 100) and then attached together, to enable other construction methods.

In the exemplary embodiment, each of the first and second arms 201, 203 is coupled to the ramp terminus 250 at the distal end 253 thereof. In other words, in the exemplary embodiment, the proximal end 282 of the first arm 201 is coupled to the distal end 253 of the ramp terminus 250, and the proximal end 286 of the second arm 203 is coupled to the distal end 253 of the ramp terminus 250. In other embodiments in which the ramp terminus 250 is included in the first and/or second arms 201, 203, each of the first and second arms 201, 203 are coupled to the anchor wire 260 at the distal end 263 thereof. That is, the proximal ends 282, 286 of each of the first and second arms 201, 203 are coupled to the anchor wire 260 at the distal end 263 thereof.

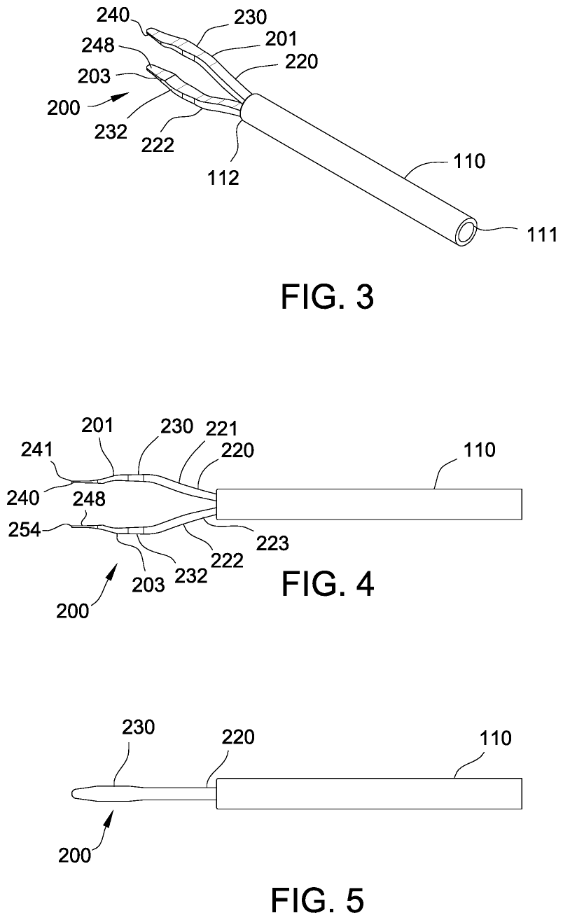

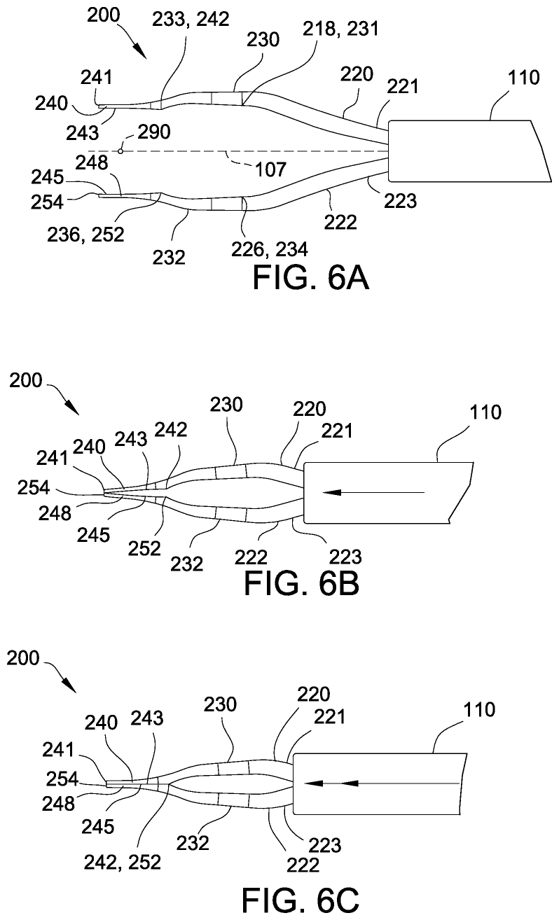

With additional reference to FIGS. 3-5, the actuation tube 110 is movable relative to the first and second ramp portions 220, 222 in the proximal and distal directions (i.e., along axis 107 shown in FIG. 1). Movement of the actuation tube 110 is controlled via the actuator 105 included in the handle 101 (shown in FIG. 1). Actuation of the actuator 105 causes the actuation tube 110 to move distally from the handle 101 and into engagement with the first and second arms 201, 203 to cause the first and second grasping platforms 240, 248 to move from an initial, open position towards a closed position. More specifically, distal movement of the of the actuation tube 110 via the actuator 105 towards the distal ends 284, 288 of the first and second arms 201, 203 causes the actuation tube 110 to engage the first and second arms 201, 203 along the ramp portions 220, 222, and causes the first and second grasping platforms 240, 248 to move from an initial, open position (shown in FIGS. 3 and 4) towards a closed position (shown in FIGS. 6C and 10). That is, distal movement of the actuation tube 110 via the actuator 105 towards the distal ends 284, 288 of the first and second arms 201, 203 causes the actuation tube 110 to engage and push upon first and second outer surfaces 221, 223 of the first and second ramp portions 220, 222, respectively, which causes the first and second ramp portions 220, 222 to bend and approach each other. Bending of the first and second ramp portions 220, 222 such that the first and second ramp portions 220, 222 approach each other, in turn, causes the first and second intermediate portions 230, 232 and the first and second grasping platforms 240, 248 to approach each other. Proximal motion of the actuation tube 110 from an actuated position (i.e., from a position in which the actuation tube 110 is engaging and pushing upon the first and second outer surfaces 221, 223 of the first and second ramp portions 220, 222) results in the first and second ramp portions 220, 222 and other features (i.e., the first and second grasping platforms 240, 248 and the first and second intermediate portions 230, 232) moving towards their initial (i.e., unbent or unflexed) positions.

FIGS. 6A, 6B, and 6C are enlarged top views of the forceps tip 200 and actuation tube 110 shown in FIG. 3. FIGS. 6A, 6B, and 6C illustrate the response of the first and second ramp portions 220, 222, the first and second intermediate portions 230, 232, and the first and second grasping platforms 240, 248 to distal motion of the actuation tube 110.

FIG. 6A illustrates the forceps tip 200 in the initial, open position (i.e., when the actuation tube 110 has not yet engaged the outer surfaces 221, 223 of the first and second ramp portions 220, 222). In the configuration shown in FIG. 6A, the gap between inner engagement surfaces 243, 245 (described further herein) of the grasping platforms 240, 248 (in the vertical direction with respect to FIG. 6A) is about 25-30 thousandths of inch (0.6-0.75 mm).

With additional reference to FIG. 6A, the first ramp portion 220 extends from a proximal end 216 (shown in FIG. 2) connected to the distal end 253 of the ramp terminus 250 (shown in FIG. 2), to a distal end 218. The first intermediate portion 230 extends from a proximal end 231 to a distal end 233, and the proximal end 231 of the first intermediate portion 230 extends from the distal end 218 of the first ramp portion 220. The first grasping platform 240 extends from a proximal end 242 to a distal end 241, and the proximal end 242 of the first grasping platform 240 extends from the distal end 233 of the first intermediate portion 230. The first grasping platform 240 includes an inner engagement surface 243 that extends arcuately from the proximal end 242 to the distal end 241.

The second ramp portion 222 extends from a proximal end 224 (shown in FIG. 2) connected to the distal end 253 of the ramp terminus 250, to a distal end 226. The second intermediate portion 232 extends from a proximal end 234 to a distal end 236, and the proximal end 234 of the second intermediate portion 232 extends from the distal end 226 of the second ramp portion 222. The second grasping platform 248 extends from a proximal end 252 to a distal end 254, and the proximal end 252 of the second grasping platform 248 extends from the distal end 236 of the second intermediate portion 232. The second grasping platform 248 includes an inner engagement surface 245 that extends arcuately from the proximal end 252 to the distal end 254.

In the exemplary embodiment, each of the inner engagement surfaces 243, 245 of the grasping platforms 240, 248 is arcuate about a transverse axis 290 extending between the inner engagement surfaces 243, 245 and perpendicular to the longitudinal axis 107 of the handle 101. That is, the transverse axis 290 runs into and out of the page in the view shown in FIG. 6A. In the exemplary embodiment, each of the inner engagement surfaces 243, 245 is concavely arcuate relative to the transverse axis 290.

Further, in the exemplary embodiment, each of the first and second ramp portions 220, 222 symmetrically diverge away from each other as each ramp portion 220, 222 extends towards its respective distal end 218, 226. Additionally, in the exemplary embodiment, the first and second arms 201, 203 are mirror images of each other. That is, the ramp portions 220, 222 are mirror images of each other, the intermediate portions 230, 232 are mirror images of each other, and the first and second grasping platforms 240, 248 are mirror images of each other. However, in other embodiments, the ramp portions 220, 222, the intermediate portions 230, 232, and the grasping platforms 240, 248 may have any suitable configuration that enables the forceps 100 to function as described herein.

FIG. 6B illustrates the forceps tip 200 positioned with the actuation tube 110 actuated to a first position in which the distal ends 241, 254 of the first and second grasping platforms 240, 248 initially engage or contact each other. In the exemplary embodiment, the first and second grasping platforms 240, 248 are configured to initially contact one another only at their respective distal ends 241, 254 along the inner engagement surfaces 243, 245 when actuated from the open position (shown in FIG. 6A) to the closed position (shown in FIGS. 6C and 10). That is, in the exemplary embodiment, the inner engagement surfaces 243, 245 of the grasping platforms 240, 248 at the distal ends 241, 254 of the grasping platforms 240, 248 make contact with each other before any other part or portion of the grasping platforms 240, 248.

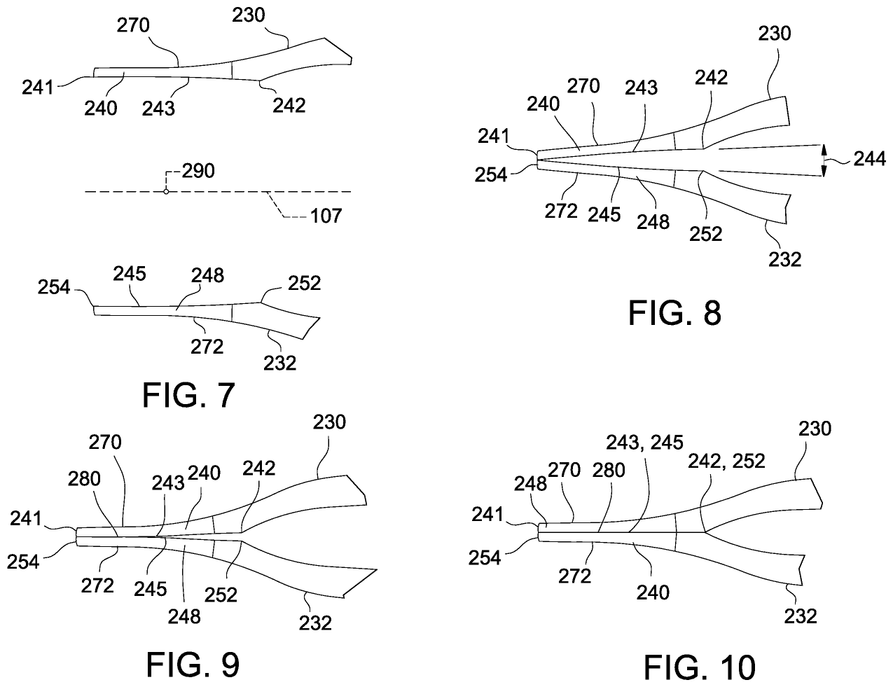

FIG. 6C illustrates the forceps tip 200 positioned with the actuation tube 110 actuated to fully articulate (i.e., close) the first and second grasping platforms 240, 248. The arrows (shown within the actuation tube 110 in FIGS. 6B and 6C) indicate the distal movement of the actuation tube 110 that enables actuation of the first and second grasping platforms 240, 248. The response of the grasping platforms 240, 248 to actuation of the actuation tube 110 is described in further detail below with reference to FIGS. 7-10.

The shape, length, and thickness of the first and second ramp portions 220, 222 may be chosen to determine the response of the forceps tip 200 to actuation of the actuation tube 110 and to determine the amount of restorative force generated by the first and second ramp portions 220, 222 to return the first and second ramp portions 220, 222 (and thus the other components of the forceps tip 200) to the relaxed, open position when relaxed by proximal motion of the actuation tube 110. The shape, length, and thickness of the first and second intermediate portions 230, 232 may be chosen to determine the stiffness of the forceps 100, the reach of the forceps 100, and visualization around the forceps 100. In the exemplary embodiment, the geometry of the first and second intermediate portions 230, 232 was chosen to maximize stiffness while still maintaining good visualization around the forceps tip 200. For example, the exemplary forceps tip 200 is configured to be very thin, which enables enhanced sight around the forceps tip 200. Additionally, in the exemplary embodiment, the first and second ramp portions 220, 222 and first and second intermediate portions 230, 232 are wider, as measured with respect to the vertical direction of FIG. 5, compared to other portions of the forceps tip 200, and the first and second ramp portions 220, 222 and first and second intermediate portions 230, 232 are constructed and configured such that the first and second ramp portions 220, 222 and the first and second intermediate portions 230, 232 do not tend to bend or flex during actuation of the first and second grasping platforms 240, 248. That is, in the exemplary embodiment, the majority of the bending and flexing (or deflecting) is concentrated in the first and second grasping platforms 240, 248, and more specifically, in the distal ends 241, 254 of the first and second grasping platforms 240, 248. Comparative forceps tips allow more bending and flexing in the comparative intermediate portions and comparative ramp portions, with the comparative grasping platforms having a much greater stiffness. In other words, comparative forceps tips do not allow for the majority of bending and flexing to be concentrated in the grasping platforms, as is the case for the exemplary forceps tip 200 (described further herein).

FIGS. 7-10 are further enlarged top views of the forceps tip 200. Specifically, FIGS. 7-10 are enlarged top views of an area near the first and second grasping platforms 240, 248 of the forceps tip 200 illustrating the response of the first and second grasping platforms 240, 248 to distal motion of the actuation tube 110. FIG. 7 shows the forceps tip 200 in the relaxed, open position (i.e., when the actuation tube 110 has not yet contacted the first and second ramp portions 220, 222), corresponding to the position shown in FIG. 6A.

As shown in FIGS. 7-10, the exemplary inner engagement surfaces 243, 245 of each grasping platform 240, 248 are shaped generally like a curved section of a cylinder with a decreasing radius of curvature between the distal ends 241, 254 and the corresponding proximal ends 242, 252 of each respective grasping platform 240, 248. That is, a radius of curvature of each of the inner engagement surfaces 243, 245 decreases from the distal end 241, 254 of the respective first or second grasping platform 240, 248 to the proximal end 242, 252 of the respective first or second grasping platform 240, 248. Such a configuration causes the inner engagement surface 243, 245 of each grasping platform 240, 248 to appear as a curve from the top view, and for the inner engagement surface 243, 245 of each grasping platform 240, 248 to be non-planar. In the exemplary embodiment, the radius of curvature of the inner engagement surface 243, 245 of each grasping platform 240, 248 starts at 0.300 inches at each distal end 241, 254 and decreases to 0.200 inches at the corresponding proximal end 242, 252. In other embodiments, a radius of curvature of 0.1 inches to 1 inch may be appropriate depending on the stiffness of the grasping platforms 240, 248, the stiffness of the intermediate portions 230, 232, and the material used to construct the components of the forceps tip 200. In some embodiments, the radius of curvature of each inner engagement surface 243, 245 of each gasping platform 240, 248 may be between 0.200 inches and 0.400 inches at the distal end 241, 254 of the respective first or second grasping platform 240, 248, and between 0.100 inches and 0.300 inches at the proximal end 242, 252 of the respective first or second grasping platform 240, 248. In yet other embodiments, the inner engagement surfaces 243, 245 may have any suitable radius of curvature and any suitable variation in their radius of curvature that enables the forceps 100 to function as described herein.

The decreasing radius of the inner engagement surface 243, 245 of each grasping platform 240, 248 from the distal end 241, 254 to the corresponding proximal end 242, 252 compensates for the increased strain in the proximal portions (i.e., portions near proximal end 242, 252 of each grasping platform 240, 248) of the grasping platforms 240, 248 as the grasping platforms 240, 248 are articulated (deflected), and allows each inner engagement surface 243, 245 of each grasping platform 240, 248 to become substantially flat when the grasping platforms 240, 248 are fully articulated (e.g., as shown in FIG. 10). When fully articulated, the inner engagement surfaces 243, 245 of the grasping platforms 240, 248 are entirely in contact or flush with one another.

The first grasping platform 240 has a thickness extending from the inner engagement surface 243 of the first grasping platform 240 to an outer surface 270 of the first grasping platform 240, and the second grasping platform 248 has a thickness extending from the inner engagement surface 245 to an outer surface 272 of the second grasping platform 248. In the exemplary embodiment, the thickness of each of the first and second grasping platforms 240, 248 increases from the distal end 241, 254 of the respective first or second grasping platform 240, 248 to the proximal end 242, 252, of the respective first or second grasping platform 240, 248. In the example embodiment, the thickness of at least a portion of each grasping platform 240, 248 is 0.001 inches. In other embodiments, the thickness of each grasping platform 240, 248 may be less than 0.005 inches, or may be in the range of 0.0005 inches to 0.005 inches. The thickness can be selected, for example, based on the desired pressure to be created between the grasping platforms 240, 248 as they are articulated and depending on the material used for construction. The thickness of the first grasping platform 240 may be the same as, or different from, the thickness of the second grasping platform 248.

The outer surface 270, 272 of each grasping platform 240, 248 generally diverges away from inner engagement surface 243, 245 of the same grasping platform 240, 248 as the outer surface 270, 272 of the grasping platform 240, 248 extends from the distal end 241, 254 to the corresponding proximal end 242, 252 of each respective grasping platform 240, 248. That is, the thinnest section (with reference to the vertical direction in FIGS. 7-10) of each grasping platform 240, 248 is at the distal end 241, 254 of each grasping platform 240, 248, with the thickness of each grasping platform 240, 248 increasing from the distal end 241, 254 to the proximal end 242, 252 of each grasping platform 240, 248. In other embodiments, the first and second grasping platforms 240, 248 may have any suitable configuration that enables the forceps 100 to functions as described herein.

As illustrated in FIGS. 7-10, the outer surface 270, 272 of each grasping platform 240 is shaped generally like a curved section of a cylinder oriented opposite to the curvature of the corresponding inner engagement surface 243, 245 of the same grasping platform 240, 248. That is, with the forceps tip 200 in the relaxed, open position (i.e., in a pre-contact configuration), the inner engagement surface 243, 245 of each grasping platform 240, 248 is curved about the transverse axis 290, while the corresponding outer surface 270, 272 of each grasping platform 240, 248 is curved opposite to the inner engagement surface 243, 245. In the exemplary embodiment shown in FIGS. 7-10, both the inner engagement surface 243, 245 and the outer surface 270, 272 of each grasping platform 240, 248 are curved opposite one another. In this embodiment, the inner engagement surfaces 243, 245 and the outer surfaces 270, 272 are concavely arcuate (or curved).

In some embodiments, the radius of curvature of the outer surface 270, 272 of each grasping platform 240, 248 is approximately 0.056 inches. In other embodiments, the radius of curvature of the outer surface 270, 272 is in the range of 0.020 inches to 1 inch. In other embodiments, the radius of curvature of the outer surface 270, 272 of each grasping platform 240, 248 may vary along the length of each grasping platform 240, 248 (i.e., between the distal end 241, 254 and the corresponding proximal end 242, 252 of each grasping platform 240, 248) depending on the desired stiffness of the grasping platforms 240, 248, the desired stiffness of the intermediate portions 230, 232, and the material used for construction. The combination of the radii of curvature of the inner engagement surface 243, 245 and the radii of curvature of the outer surface 270, 272 of each grasping platform 240, 248 along with the thickening of each grasping platform 240, 248 from the distal end 241, 254 to the corresponding proximal end 242, 252 determines the flexibility (i.e., the articulation response to actuation) of the grasping platforms 240, 248. In some embodiments, the radii of curvature of the inner engagement surfaces 243, 245 of the first and second grasping platforms 240, 248 may be the same and/or the radii of curvature of the outer surfaces 270, 272 of the first and second grasping platforms 240, 248 may be the same. In other embodiments, the radii of curvature of the inner engagement surfaces 243, 245 of the first and second grasping platforms 240, 248 are different from each other and/or the radii of curvature of the outer surfaces 270, 272 of the first and second grasping platforms 240, 248 are different from each other. In any embodiment, the inner engagement surfaces 243, 245 of the first and second grasping platforms 240, 248 and the outer surfaces 270, 272 of the first and second grasping platforms 240, 248 may have any suitable configuration that enables the forceps 100 to function as described herein.

FIG. 8 shows the forceps tip 200 positioned with the actuation tube 110 actuated just enough to cause the distal ends 241, 254 of the grasping platforms 240, 248 to contact each other (i.e., to cause initial contact between the grasping platforms 240, 248, as shown in FIG. 6B). That is, in the exemplary embodiment, the inner engagement surfaces 243, 245 of the grasping platforms 240, 248 at the distal ends 241, 254 of the grasping platforms 240, 248 make contact with each other before any other part or portion of the grasping platforms 240, 248. In the exemplary embodiment shown in FIG. 8, the inner engagement surfaces 243, 245 of the grasping platforms 240, 248 form a wedge-shaped gap 244 with the widest section of the gap 244 oriented or facing proximally. The wedge-shaped gap 244 has an angle formed by the inner engagement surfaces 243, 245. In some embodiments, the angle of the wedge-shaped gap 244 is in the range of 3 degrees to 15 degrees. In one particular embodiment, the angle of the wedge-shaped gap is 7 degrees. The angle of the wedge-shaped gap 244 can vary depending on the flexibility of the grasping platforms 240, 248, the desired pressure created between the grasping platforms 240, 248 as they are articulated, and the total amount of desired articulation of the grasping platforms 240, 248.

FIG. 9 shows the forceps tip 200 positioned with the actuation tube 110 actuated to partially articulate the grasping platforms 240, 248. The first and second grasping platforms 240, 248 are configured to deflect such that a contact or interface area 280 between the inner engagement surfaces 243, 245 increases from the respective distal ends 241, 254 of the first and second grasping platforms 240, 248 to the respective proximal ends 242, 252 of the first and second grasping platforms 240, 248 as the first and second grasping platforms 240, 248 move towards the closed position. As the actuation tube 110 is actuated from its position in FIG. 6B to its position in FIG. 6C, pressure builds on the distal ends 241, 254 of the grasping platforms 240, 248. This causes the radius of curvature of the inner engagement surfaces 243, 245 of the grasping platforms 240, 248 to increase and the radius of curvature of the outer surfaces 270, 272 of the grasping platforms 240, 248 to decrease. In other words, the grasping platforms 240, 248 begin to flex (or bend or deflect) and flatten out. During this process, distal portions (i.e., medial portions adjacent to distal ends 241, 254 of the grasping platforms 240, 248) of the inner engagement surfaces 243, 245 of the grasping platforms 240, 248 contact each other to increase the contact area 280. The force developed from the actuation of the grasping platforms 240, 248 is spread across the contact area 280. If the inner engagement surface 243, 245 of each grasping platform 240, 248 were flat when in the relaxed, open configuration, rather than curved, the pressure developed from the flexing of the grasping platforms 240, 248 would concentrate on the most proximal portion of the contact area 280. The curve of the inner engagement surface 243, 245 of each grasping platform 240, 248 helps to evenly spread the force over a larger contact area 280 while still maintaining pressure on the distal ends 241, 254 of the grasping platforms 240, 248. This is advantageous when only the distal end 115 (shown in FIG. 2) of the forceps tip 200 (i.e., the distal ends 241, 254 of the grasping platforms 240, 248) engages tissue. Relief of pressure on the tip (i.e., distal end 115) of the forceps 200 causes the tissue to slip from the grasp of the forceps 100 easily. If the grasping platforms 240, 248 are too stiff, then the force is concentrated in a small area at the distal ends 241, 254 of the grasping platforms 240, 248. Because tissue is only able to withstand a limited amount of pressure, the total force that the forceps tip 200 can apply to the tissue is limited by the concentration of pressure or stress. Balance of the radii of the inner engagement surface 243, 245 of each grasping platform 240, 248 and the stiffness of each grasping platform 240, 248 allows for a desirable amount of pressure to be applied and maintained on the distal end 241, 254 of each grasping platform 240, 248. As additional force is applied between the grasping platforms 240, 248, the grasping platforms 240, 248 further articulate to increase the contact area 280 between the inner engagement surfaces 243, 245 of the grasping platforms 240, 248, which enables the larger contact area 280 to spread the grasping load (i.e., force) across a larger area of the tissue to help avoid structural failure of the tissue. In some embodiments, the contact or interface area 280 between the inner engagement surfaces 243, 245 continuously increases from their respective distal ends 241, 254 to their respective proximal ends 242, 252 as the first and second grasping platforms 240, 248 move towards the closed position due to deflection or articulation of the first and second grasping platforms 240, 248.

FIG. 10 shows the forceps tip 200 positioned with the actuation tube 110 actuated to fully articulate or close the grasping platforms 240, 248, corresponding to FIG. 6C. As the grasping platforms 240, 248 are further actuated from the configuration shown in FIG. 9, the radius of the inner engagement surface 243, 245 of each grasping platform 240, 248 continues to increase and the radius of the outer surface 270, 272 of each grasping platform 240, 248 continues to decrease. As the grasping load continues to build on the inner engagement surface 243, 245 of each grasping platform 240, 248, the contact area 280 grows as each grasping platform 240, 248 “rolls” proximally, until contact is made between the proximal end 242, 252 of each grasping platform 240, 248 at which point the contact area 280 becomes a maximum contact area 280. Once contact is made between the proximal end 242, 252 of each grasping platform 240, 248 the contact area 280 can no longer grow, and any additional grasping load will be distributed over the maximum contact area 280.

### Comparative Examples

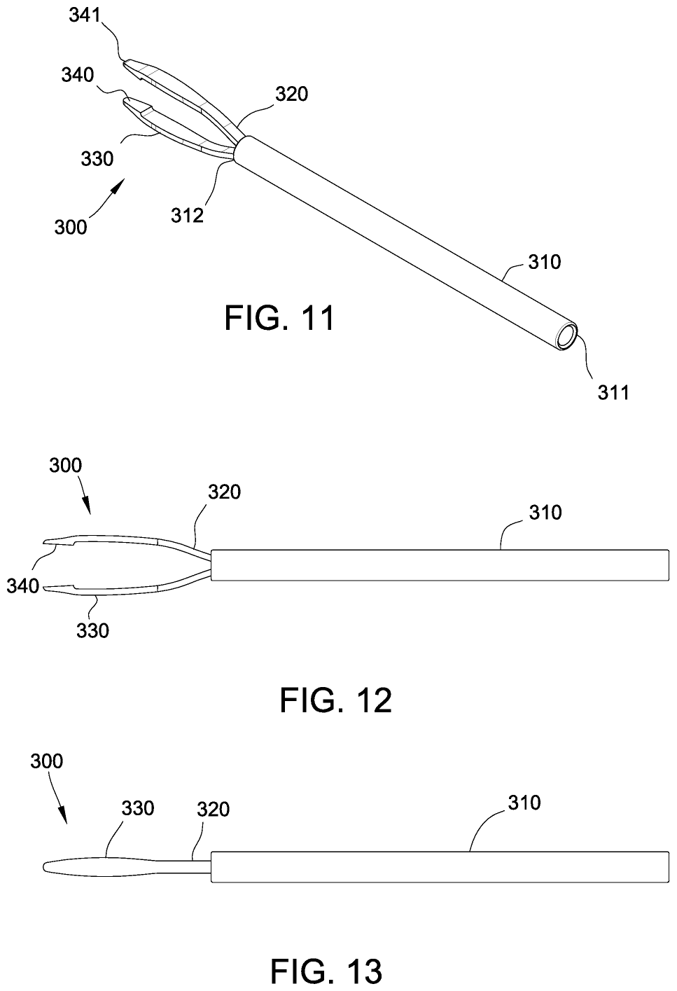

FIGS. 11-25 show different views of comparative forceps tips. In FIGS. 11-25, like components are labeled with like reference numbers. For example, in FIG. 11, comparative forceps tip 300 is shown as including two comparative grasping platforms 340. However, in FIG. 11, only one comparative grasping platform 340 is labeled.

FIG. 11 is a perspective view of a comparative forceps tip 300 including an actuation tube 310. The actuation tube 310 extends from a proximal end 311, to a distal end 312. The forceps tip 300 includes ramp portions 320, intermediate portions 330, and grasping platforms 340. The grasping platforms 340 include distal ends 341.

FIG. 12 and FIG. 13 are top and side views, respectively, of the forceps tip 300 shown in perspective in FIG. 11. The actuation tube 310 is movable relative to ramp portions 320 in a proximal direction and a distal direction via an actuation handle (not shown). Distal movement of the actuation tube 310 towards the distal ends 341 of the grasping platforms 340 causes the ramp portions 320 to bend and approach each other. This, in turn, causes the grasping platforms 340 to approach each other.

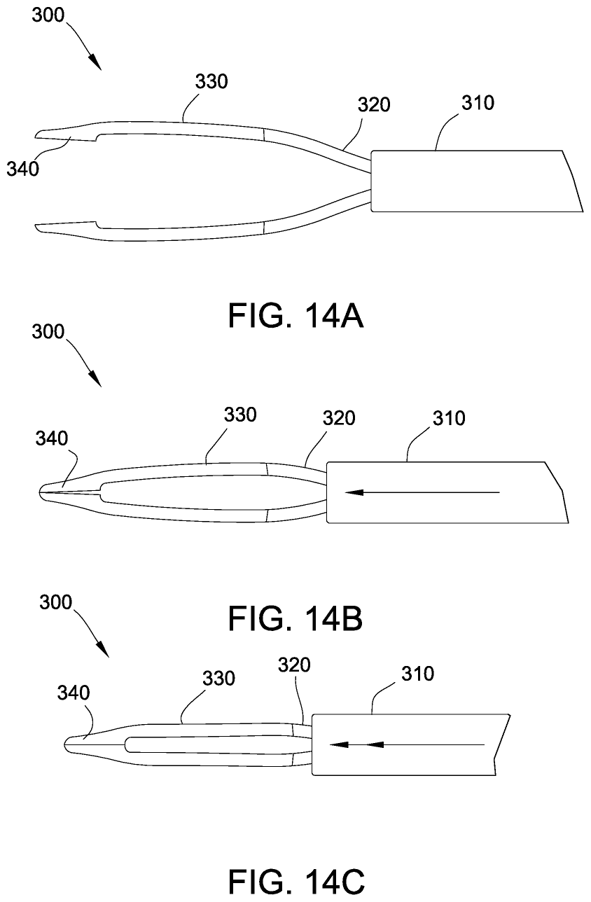

FIGS. 14A, 14B, and 14C illustrate the response of the ramp portions 320, the intermediate portions 330, and the grasping platforms 340 to distal motion of the actuation tube 310. Proximal motion of the actuation tube 310 from an actuated position results in the ramp portions 320 and other features to return to their rest positions (i.e., to the configuration shown in FIG. 14A). The shape and length of the intermediate portions 330 was chosen to provide some flexibility during actuation, as can be seen in the closed position (shown in FIG. 14C).

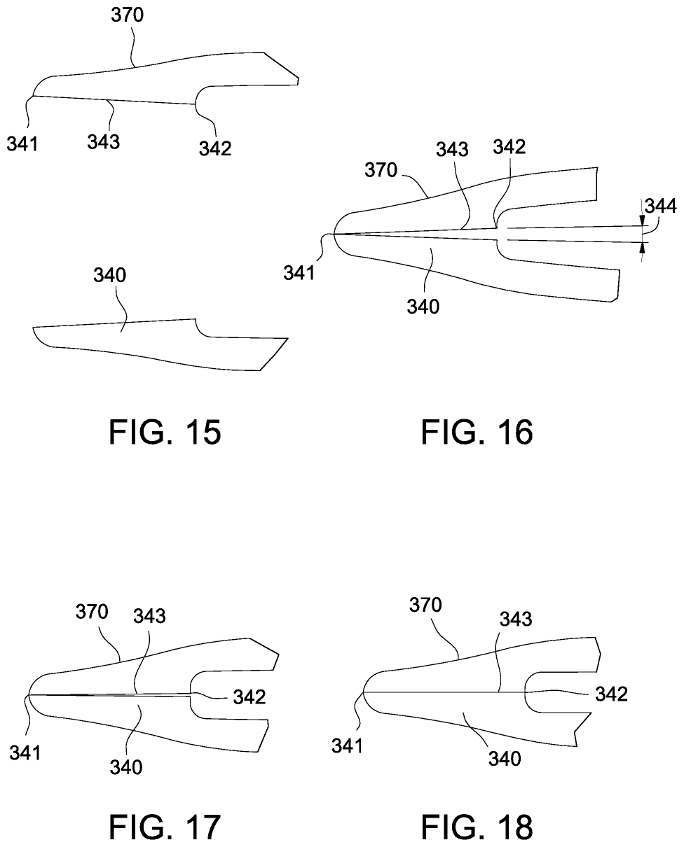

FIGS. 15-18 are top views of an enlarged area near the grasping platforms 340 of the forceps tip 300 illustrating the response of the grasping platforms 340 to distal motion of actuation tube 310 and subsequent movement of the ramp portions 320 and the intermediate portions 330. FIG. 15 shows the forceps tip 300 in the relaxed, open position. As shown in FIGS. 15-18, the grasping platforms 340 include the distal ends 341, proximal ends 342, inner engagement surfaces 343, and outer surfaces 370. The inner engagement surfaces 343 of the grasping platforms 340 are flat. In other words, the inner engagement surfaces 343 of the grasping platforms 340 each form a plane. The outer surfaces 370 of the grasping platforms 340 each define the thickness of each grasping platform 340. The outer surfaces 370 of the grasping platforms 340 generally diverge away from the corresponding inner engagement surface 343 of the grasping platforms 340, with the thinnest section of the grasping platforms 340 at the distal ends 341 of each grasping platform 340. The designed thickness of the grasping platforms 340 causes the grasping platforms 340 to be stiff. The comparative grasping platforms 340 flex a negligible amount as force is applied between the grasping platforms 340.

FIG. 16 shows the forceps tip 300 in a position with the actuation tube 310 actuated enough to cause distal ends 341 of the grasping platform 340 to contact each other. The inner engagement surfaces 343 of the grasping platforms 340 form a wedge-shaped gap 344 of approximately 4 degrees, with the widest section of the gap 344 oriented proximal. The wedge angle can range from 2 degrees to 10 degrees depending on the flexibility of the intermediate portions 330 and the desired amount of pressure to be applied to the tissue via the grasping platforms 340.

FIG. 17 shows the forceps tip 300 in a position with the actuation tube 310 actuated to partially close the grasping platforms 340. As the actuation tube 310 is actuated from its position in FIG. 16 to its position in FIG. 17, pressure builds on the distal ends 341 of the grasping platforms 340. This causes the grasping platforms 340 to pivot (i.e., not bend or flex) around the distal ends 341 as the intermediate portions 330 flex, and the wedge-shaped gap 344 formed by the inner engagement surfaces 343 of the grasping platforms 340 narrows. The pressure developed on the grasping platforms 340 is concentrated in a small area at the distal ends 341 of the grasping platforms 340. In the configuration of the comparative forceps tip 300, the stiffness of the intermediate portions 330 must be adjusted to keep the pressure in the concentrated area from exceeding the structural strength of the tissue.

FIG. 18 shows the forceps tip 300 in a position with the actuation tube 310 actuated to fully close the grasping platforms 340. As the actuation tube 310 is actuated from its position in FIG. 17 to its position shown in FIG. 18, pressure continues to build on the grasping platforms 340, and the contact area remains concentrated at the distal ends 341 of the grasping platforms 340 until the grasping platforms 340 pivot enough to close the wedge-shaped gap 344 and to bring the proximal ends 342 of the grasping platforms 340 in contact. Once the proximal ends 342 of the grasping platforms 340 are in contact, the force between the grasping platforms 340 spreads over the entire inside contact area of the grasping platforms 340.

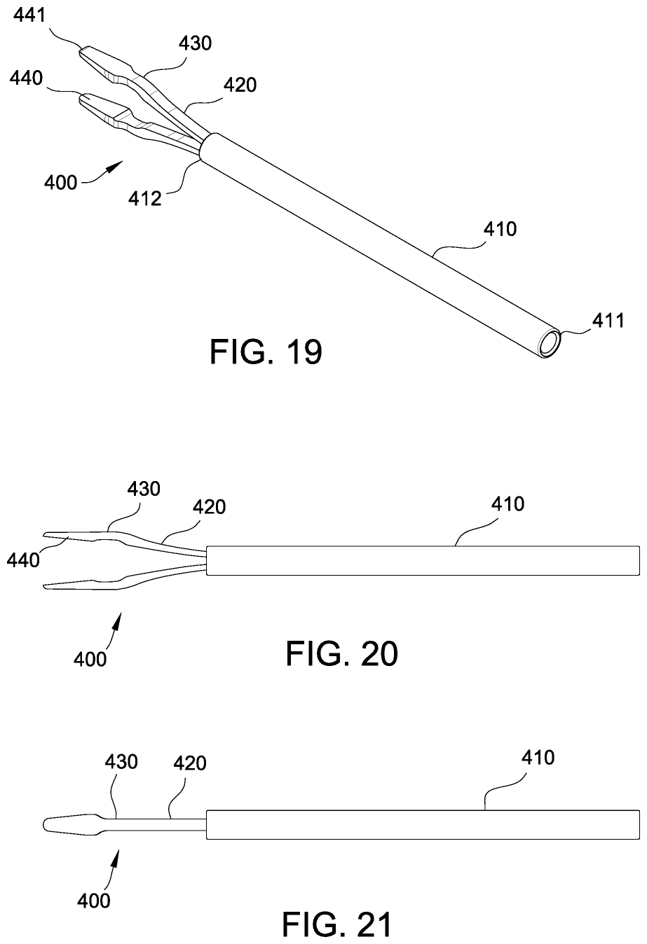

FIG. 19 is a perspective view of another comparative forceps tip 400 including an actuation tube 410. The actuation tube 410 extends from a proximal end 411 to a distal end 412. The actuation tube 410 includes ramp portions 420, intermediate portions 430, and grasping platforms 440. The grasping platforms include distal ends 441.

FIGS. 20 and 21 are top and side views, respectively, of the forceps tip 400 shown in FIG. 19. The actuation tube 410 is movable relative to the ramp portions 420 in a proximal direction and a distal direction via an actuation handle (not shown). Distal movement of the actuation tube 410 towards the distal ends 441 of the grasping platforms 440 causes the ramp portions 420 to bend and approach each other. This, in turn, causes the grasping platforms 440 to approach each other.

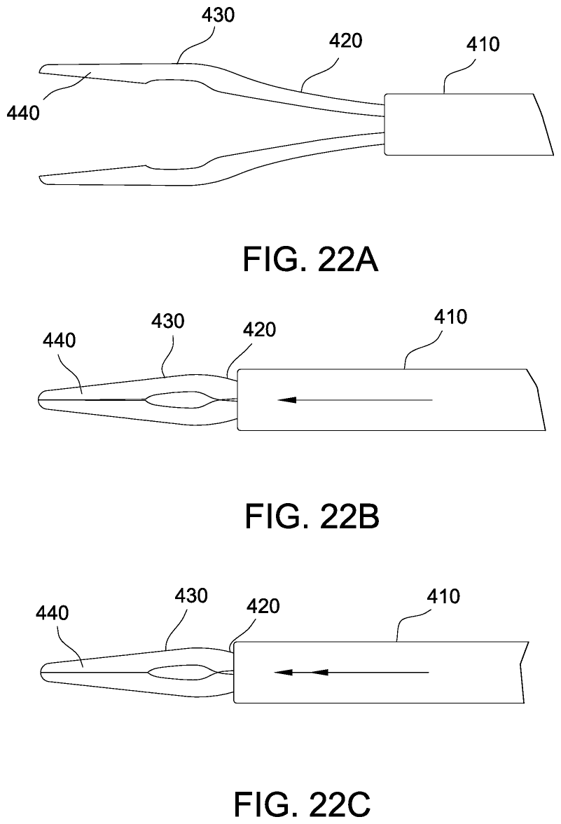

FIGS. 22A, 22B, and 22C illustrate the response of the ramp portions 420, the intermediate portions 430, and the grasping platforms 440 to distal motion of actuation tube 410. Proximal motion of the actuation tube 410 from an actuated position results in the ramp portions 420 and other features to return to their rest positions (i.e., to the configuration shown in FIG. 22A). The shape and length of the intermediate portions 430 was chosen to be stiff. That is, the intermediate portions 430 resist bending and also resist pivoting of the grasping platforms 440 when pressure is applied to the grasping platforms 440.

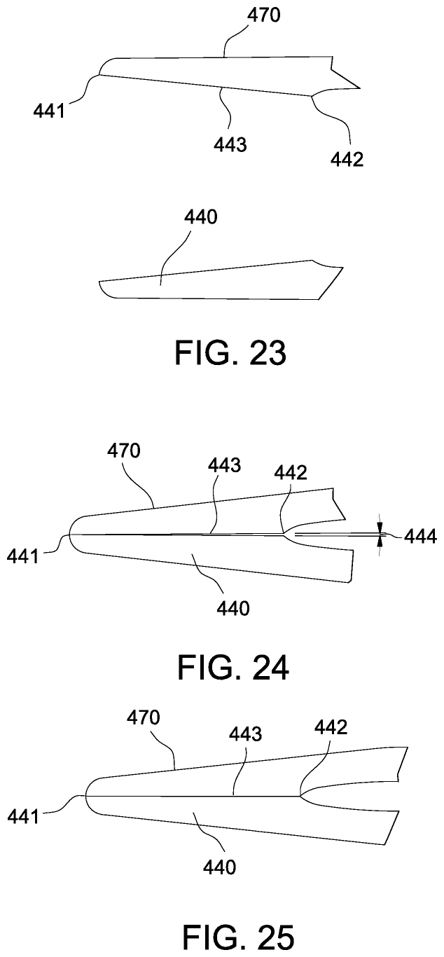

FIGS. 23-25 are top views of an enlarged area near the grasping platforms 440 of forceps tip 400 illustrating the response of the grasping platforms 440 to distal motion of the actuation tube 410 and subsequent movement of the ramp portions 420 and the intermediate portions 430.

FIG. 23 shows the forceps tip 400 in the relaxed, open position. The grasping platforms 440 include the distal ends 441, proximal ends 442, inner engagement surfaces 443, and outer surfaces 470. The inner engagement surface 443 of each grasping platform 440 is flat. That is, the inner engagement surface of each grasping platform 440 forms a plane. The outer surface 470 of each grasping platform 440 defines the thickness of the grasping platform 440. The outer surface 470 of each grasping platform generally diverges away from the corresponding inner engagement surface 443 of the grasping platform 440, with the thinnest section of the grasping platforms 440 at the distal ends 441. The thickness of the grasping platforms 440 causes each grasping platform 440 to be stiff. That is, each grasping platform 440 flexes a negligible amount as pressure is applied between the grasping platforms 440.

FIG. 24 shows the forceps tip 400 in a position with the actuation tube 410 actuated enough to cause the distal ends 441 of each grasping platform 440 to contact each other. In such a configuration, the inner engagement surfaces 443 of the grasping platforms 440 form a wedge-shaped gap 444 of 1 degree or less, with the widest section of the gap 444 oriented proximal. The wedge angle is kept to a minimum because the grasping platforms 440 and the intermediate portions 430 are stiff.

FIG. 25 shows the forceps tip 400 in a position with the actuation tube 410 actuated to fully close the grasping platforms 440. As the actuation tube 410 is actuated from its position in FIG. 24 to its position shown in FIG. 25, some pressure builds on the grasping platforms 440 that is concentrated at the distal ends 441 of the grasping platforms 440. The pressure does not build too high at the distal ends 441 of the grasping platforms 440 because the proximal ends 442 of the grasping platforms 440 come in contact simultaneously with the actuation tube 410 reaching its maximum distal movement. In this comparative example, calibration of the stopping position (i.e., the point of maximum distal movement) of the actuation tube 410 is critical to control the pressure developed between the grasping platforms 440. Because of the stiffness of the grasping platforms 440 and the stiffness of the intermediate portions 430, a minor change in the stopping position of the actuation tube 410 causes the pressure developed between the grasping platforms 440 to vary from very low levels (or none at all) to very high levels. The developed pressure can also vary a great amount with the thickness of tissue that is grasped by the grasping platforms 440.

Although specific features of various embodiments of the disclosure may be shown in some drawings and not in others, this is for convenience only. In accordance with the principles of the disclosure, any feature of a drawing may be referenced and/or claimed in combination with any feature of any other drawing.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A forceps comprising:
a handle extending along a longitudinal axis and comprising an actuator;
an actuation tube operatively coupled to the actuator; and
a forceps tip comprising:
a first arm comprising a first grasping platform extending from a proximal end to a distal end, the first grasping platform comprising a first inner engagement surface that extends arcuately from the proximal end to the distal end; and
a second arm comprising a second grasping platform extending from a proximal end to a distal end, the second grasping platform comprising a second inner engagement surface that extends arcuately from the proximal end to the distal end, wherein each of the first and second engagement surfaces is arcuate about a transverse axis extending between the first and second engagement surfaces and perpendicular to the longitudinal axis of the handle;
wherein actuation of the actuator causes the actuation tube to move distally from the handle and into engagement with the first and second arms to cause the first and second grasping platforms to move from an initial, open position towards a closed position;
wherein the first and second grasping platforms are configured to initially contact one another only at their respective distal ends along the first and second engagement surfaces when actuated from the open position to the closed position; and
wherein each of the first and second grasping platforms is configured to deflect such that a contact area between the first and second engagement surfaces increases from their respective distal ends to their respective proximal ends as the first and second grasping platforms move towards the closed position.

2. The forceps of claim 1, wherein each of the first and second engagement surfaces is shaped as a curved section of a cylinder.

3. The forceps of claim 1, wherein a radius of curvature of each of the first and second engagement surfaces decreases from the distal end of the respective first or second grasping platform to the proximal end of the respective first or second grasping platform.

4. The forceps of claim 3, wherein the radius of curvature of each of the first and second engagement surfaces is between 0.200 inches and 0.400 inches at the distal end of the respective first or second grasping platform, and between 0.100 inches and 0.300 inches at the proximal end of the respective first or second grasping platform.

5. The forceps of claim 1, wherein the first grasping platform has a thickness extending from the first inner engagement surface to a first outer surface, wherein the second grasping platform has a thickness extending from the second inner engagement surface to a second outer surface, and wherein the thickness of each of the first and second grasping platforms increases from the distal end of the respective first or second grasping platform to the proximal end of the respective first or second grasping platform.

6. The forceps of claim 5, wherein the thickness of each of the first and second grasping platforms is less than 0.005 inches.

7. The forceps of claim 5, wherein each of the first and second outer surfaces is concavely arcuate.

8. The forceps of claim 1, wherein the forceps tip is formed by one of electric discharge machining and laser cutting.

9. The forceps of claim 1, wherein the forceps tip further comprises an anchor wire extending through the actuation tube and fixed to the handle, wherein each of the first and second arms is coupled to the anchor wire.

10. The forceps of claim 9, wherein the anchor wire and the first and second arms are integrally formed from a single piece of material.

11. The forceps of claim 1, wherein the first and second arms are mirror images of each other.

12. The forceps of claim 1, wherein the first grasping platform and the second grasping platform are mirror images of each other.

13. A forceps comprising:
a handle extending along a longitudinal axis and comprising an actuator; and
a forceps tip operatively coupled to the actuator, the forceps tip comprising:
a first arm comprising a first grasping platform extending from a proximal end to a distal end, the first grasping platform comprising a first inner engagement surface that extends arcuately from the proximal end to the distal end; and
a second arm comprising a second grasping platform extending from a proximal end to a distal end, the second grasping platform comprising a second inner engagement surface that extends arcuately from the proximal end to the distal end;
wherein actuation of the actuator causes the first and second grasping platforms to move from an initial, open position towards a closed position; and
wherein each of the first and second grasping platforms is configured to deflect such that a contact area between the first and second engagement surfaces continuously increases between their respective distal ends and their respective proximal ends as the first and second grasping platforms move towards the closed position.

14. The forceps of claim 13, wherein each of the first and second engagement surfaces is shaped as a curved section of a cylinder.

15. The forceps of claim 13, wherein the first and second grasping platforms are configured to initially contact one another only at their respective distal ends along the first and second engagement surfaces when actuated from the open position to the closed position.

16. The forceps of claim 13, wherein a radius of curvature of each of the first and second engagement surfaces decreases from the distal end of the respective first or second grasping platform to the proximal end of the respective first or second grasping platform.

17. The forceps of claim 13, wherein the first grasping platform has a thickness extending from the first inner engagement surface to a first outer surface, wherein the second grasping platform has a thickness extending from the second inner engagement surface to a second outer surface, and wherein the thickness of each of the first and second grasping platforms increases from the distal end of the respective first or second grasping platform to the proximal end of the respective first or second grasping platform.

18. The forceps of claim 13 further comprising an actuation tube operatively coupled to the actuator, wherein the forceps tip is operatively coupled to the actuator through the actuation tube, wherein the forceps tip further comprises an anchor wire extending through the actuation tube and fixed to the handle, wherein each of the first and second arms is coupled to the anchor wire.

19. The forceps of claim 18, wherein the anchor wire and the first and second arms are integrally formed from a single piece of material.

20. A method of operating forceps, the method comprising:
providing a forceps comprising:
a handle extending along a longitudinal axis and comprising an actuator;
an actuation tube operatively coupled to the actuator; and
a forceps tip comprising:
a first arm comprising a first grasping platform extending from a proximal end to a distal end, the first grasping platform comprising a first inner engagement surface that extends arcuately from the proximal end to the distal end; and
a second arm comprising a second grasping platform extending from a proximal end to a distal end, the second grasping platform comprising a second inner engagement surface that extends arcuately from the proximal end to the distal end, wherein each of the first and second engagement surfaces is arcuate about a transverse axis extending between the first and second engagement surfaces and perpendicular to the longitudinal axis of the handle; and
actuating the actuator such that the actuation tube moves distally from the handle and into engagement with the first and second arms to cause the first and second grasping platforms to move from an initial, open position towards a closed position;
wherein actuating the actuator causes the first and second grasping platforms to initially contact one another only at their respective distal ends along the first and second engagement surfaces when actuated from the open position to the closed position; and
wherein actuating the actuator causes each of the first and second grasping platforms to deflect such that a contact area between the first and second engagement surfaces increases from their respective distal ends to their respective proximal ends as the first and second grasping platforms move towards the closed position.
